(19) United States
(12) Patent Application Publication
(10) Pub. No.: US 2009/0131410 A1
(43) Pub. Date: May 21, 2009

(54) 3-AZABICYCLOOCTANE DERIVATIVES AS MUSCARINIC RECEPTOR ANTAGONISTS

(76) Inventors: Naresh Kumar, Gurgaon (IN); Kirandeep Kaur, Gurgaon (IN); Suman Gupta, Gurgaon (IN); Anita Chugh, New Delhi (IN); Mohammad Salman, Princeton, NJ (US); Raj Kumar Shirumalla, New Delhi (IN); Shivani Malhotra, New Delhi (IN)

Correspondence Address: RANBAXY INC., 600 COLLEGE ROAD EAST, SUITE 2100, PRINCETON, NJ, 08540, US

(21) Appl. No.: 12/089,150

(22) PCT Filed: Oct. 5, 2006
(86) PCT No.: PCT/IB2006/053650

§ 371 Date: Sep. 23, 2008

(30) Foreign Application Priority Data
Oct. 5, 2005 (IN) 2670/DEL/2005

(51) Int. Cl. A61K 31/55; C07D 223/14; A61P 3/10

(52) U.S. Cl. 514/216; 540/593

(57) ABSTRACT

Provided are muscarinic receptor antagonists, which can be useful in treating various diseases of the respiratory, urinary and gastrointestinal systems mediated through muscarinic receptors. Also provided are processes for preparing compounds described herein, pharmaceutical compositions thereof, and methods for treating diseases mediated through muscarinic receptors.

### FIELD OF THE INVENTION

Provided are muscarinic receptor antagonists, which can be useful in treating various diseases of the respiratory, urinary and gastrointestinal systems mediated through muscarinic receptors. Also provided are processes for preparing compounds described herein, pharmaceutical compositions thereof, and methods for treating diseases mediated through muscarinic receptors.

### BACKGROUND OF THE INVENTION

Physiological effects elicited by the neurotransmitter acetylcholine are mediated through its interaction with two major classes of acetylcholine receptors—the nicotinic and muscarinic acetylcholine receptors. Muscarinic receptors belong to the superfamily of G-protein coupled receptors and five molecularly distinct subtypes are known to exist (M₁, M₂, M₃, M₄ and M₅).

These receptors are widely distributed on multiple organs and tissues and are critical to the maintenance of central and peripheral cholinergic neurotransmission. The regional distribution of these receptor sub-types in the brain and other organs has been documented (for example, the M₁ subtype is located primarily in neuronal tissues such as cereberal cortex and autonomic ganglia, the M₂ subtype is present mainly in the heart and bladder smooth muscle, and the M₃ subtype is located predominantly on smooth muscle and salivary glands (Nature, 323, p. 411 (1986); Science, 237, p. 527 (1987)).

Curr. Opin. Chem. Biol, 3, p. 426 (1999) and Trends in Pharmacol. Sci., 22, p. 409 (2001) by Eglen et al. describes the biological potentials of modulating muscarinic receptor subtypes by ligands in different disease conditions, such as Alzheimer's disease, pain, urinary disease condition, chronic obstructive pulmonary disease, and the like.

The pharmacological and medical aspects of the muscarinic class of acetylcholine agonists and antagonists have been described. Molecules, 6, p. 142(2001). Recent developments on the role of different muscarinic receptor subtypes using different muscarinic receptor of knock out mice. Birdsall et al, Trends in Pharmacol. Sci., 22, p. 215 (2001)

Almost all smooth muscle express a mixed population of M₂ and M₃ receptors. Although the M₂-receptors are the predominant cholinoreceptors, the smaller population of M₃-receptors appears to be the most functionally important as they mediate the direct contraction of these smooth muscles. Muscarinic receptor antagonists are known to be useful for treating various medical conditions associated with improper smooth muscle function, such as overactive bladder syndrome, irritable bowel syndrome and chronic obstructive pulmonary disease. However the therapeutic utility of antimuscarinics has been limited by poor tolerability as a result of treatment related, frequent systemic adverse events such as dry mouth, constipation, blurred vision, headache, somnolence and tachycardia. Thus, there exists a need for novel muscarinic receptor antagonists that demonstrate target organ selectivity.

WO 04/005252 discloses azabicyclo derivatives described as musacrinic receptor antagonists. WO 04/004629, WO 04/052857, WO 04/067510, WO 04/014853, WO 04/014363 discloses 3,6-disubstituted azabicyclo[3.1.0]hexane derivatives described as useful muscarinic receptor antagonists. WO2004/056811 discloses flaxavate derivatives as muscarinic receptor antagonists. WO2004/056810 discloses xanthene derivatives as muscarinic receptor antagonists. WO2004/056767 discloses 1-substituted-3-pyrrolidine derivatives as muscarinic receptor antagonists. WO2004/089363, WO2004/089898, WO04069835, WO2004/089900 and WO2004089364 disclose substituted azabicyclohexane derivatives as muscarinic receptor antagonists. WO2005/026121 and WO2005/026121 discloses process for preparing azabicyclohexane derivatives. WO2006/018708 discloses pyrrolidine derivatives as muscarinic receptor antagonists. WO06/054162, WO06/016245, WO06/016345, WO06/05282 and WO2006/35303 disclose azabicyclo derivatives as muscarinic receptor antagonists. WO06/032994 discloses amine derivatives as muscarinic receptor antagonists.

J. Med. Chem., 44, p. 984 (2002) discloses cyclohexylmethylpiperidinyl-triphenylpropioamide derivatives as selective M₃ antagonist discriminating against the other receptor subtypes. J. Med. Chem., 36, p. 610 (1993) discloses the synthesis and antimuscarinic activity of some 1-cycloalkyl-1-hydroxy-1-phenyl-3-(4-substituted piperazinyl)-2-propanones and related compounds. J. Med. Chem., 34, p. 3065 (1991) discloses analogues of oxybutynin, synthesis and antimuscarinic activity of some substituted 7-amino-1-hydroxy-5-heptyn-2-ones and related compounds. Bio-Organic Medicinal Chemistry Letters, 15, p. 2093 (2005) discloses synthesis and activity of analogues of Oxybutynin and Tolterodine. Chem. Pharm. Bull, 53(4), 437, 2005 discloses synthesis and activity of 2-aminothiazole-4-carboxamides.

In view of the above, however, there remains a need for novel muscarinic receptor antagonists that can be useful in treating disease states associated with improper smooth muscle function and respiratory disorders.

### SUMMARY OF THE INVENTION

In one aspect, provided are muscarinic receptor antagonists, which can be useful as safe and effective therapeutic or prophylactic agents for treating various diseases of the respiratory, urinary and gastrointestinal systems. Also provided are processes for synthesizing such compounds.

In another aspect, pharmaceutical compositions containing such compounds are provided together with acceptable carriers, excipients or diluents which can be useful for treating various diseases of the respiratory, urinary and gastrointestinal systems.

The enantiomers, diastereomers, N-oxides, polymorphs, pharmaceutically acceptable salts and pharmaceutically acceptable solvates of these compounds, metabolites having the same type of activity, as well as pharmaceutical compositions comprising such compounds described herein and with one or more pharmaceutically acceptable carriers, excipients or diluents.

Other aspects will be set forth in the description which follows, and in part will be apparent from the description or may be learnt by the practice of the invention.

Thus in one aspect, provided are compounds having the structure of Formula I:

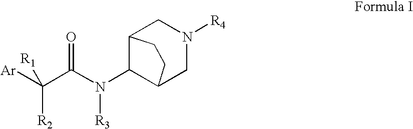

wherein
Ar can be aryl or heteroaryl;
R₁ can be hydrogen, hydroxy, alkyl, halogen or alkoxy;
R₂ can be cycloalkyl, aryl or heteroaryl;
R₃ can be hydrogen or alkyl;
R₄ can be alkyl, alkenyl, aralkyl or heteroarylalkyl;
or a pharmaceutically acceptable salt, pharmaceutically acceptable solvate, enantiomer, diastereomer, polymorph or N-oxide thereof.

In another aspect, provided are compounds selected from:
N-(3-benzyl-3-azabicyclo[3.2.1]oct-8-yl)-N-methyl-2,2-diphenylacetamide (Compound No. 1),
N-3-azabicyclo[3.2.1]oct-8-yl-N-methyl-2,2-diphenylacetamide (Compound No. 2),
N-(3-benzyl-3-azabicyclo[3.2.1]oct-8-yl)-2-hydroxy-N-methyl-2,2-diphenylacetamide (Compound No. 3),
N-(3-benzyl-3-azabicyclo[3.2.1]oct-8-yl)-N-methyl-2,2-diphenylpropanamide (Compound No. 4),
(Exo or Endo) N-(3-benzyl-3-azabicyclo[3.2.1]oct-8-yl)-2-cyclopentyl-2-hydroxy-2-(4-methylphenyl)acetamide (Compound No. 5),
(Exo or Endo) N-(3-benzyl-3-azabicyclo[3.2.1]oct-8-yl)-2-cyclopentyl-2-hydroxy-2-(4-methylphenyl)acetamide (Compound No. 6),
N-(3-benzyl-3-azabicyclo[3.2.1]oct-8-yl)-2-hydroxy-2-(4-methoxyphenyl)-2-cyclopentylacetamide (Compound No. 7),
N-(3-benzyl-3-azabicyclo[3.2.1]oct-8-yl)-2-cyclopentyl-2-hydroxy-2-(4-methoxyphenyl)acetamide (Compound No. 8),
N-3-azabicyclo[3.2.1]oct-8-yl-2-cyclopentyl-2-hydroxy-2-(4-methoxyphenyl)acetamide (Compound No. 9),
N-(3-benzyl-3-azabicyclo[3.2.1]oct-8-yl)-2-chloro-N-methyl-2,2-diphenylacetamide (Compound No. 10),
N-3-azabicyclo[3.2.1]oct-8-yl-2-cyclopentyl-2-hydroxy-2-(4-methylphenyl)acetamide (Compound No. 11),
N-3-azabicyclo[3.2.1]oct-8-yl-2-hydroxy-N-methyl-2,2-diphenylacetamide (Compound No. 12),
(Exo or Endo) N-(3-benzyl-3-azabicyclo[3.2.1]oct-8-yl)-2,2-diphenylacetamide (Compound No. 13),
(Exo or Endo) N-(3-benzyl-3-azabicyclo[3.2.1]oct-8-yl)-2,2-diphenylacetamide (Compound No. 14),
2-Hydroxy-N-methyl-N-[3-(4-methylpent-3-en-1-yl)-3-azabicyclo[3.2.1]oct-8-yl]-2,2-diphenylacetamide (Compound No. 15),
N-{3-[2-(1,3-benzodioxol-5-yl)ethyl]-3-azabicyclo[3.2.1]oct-8-yl}-2-hydroxy-N-methyl-2,2-diphenylacetamide (Compound No. 16),
N-(3-benzyl-3-azabicyclo[3.2.1]oct-8-yl)-2-hydroxy-2-phenyl-2-(2-thienyl)acetamide (Compound No. 17).

In yet another aspect, provided are pharmaceutical compositions comprising a therapeutically effective amount of a compound described herein or a pharmaceutically acceptable salt, pharmaceutically acceptable solvate, enantiomer, diastereomer, polymorph or N-oxide thereof and one or more pharmaceutically acceptable carriers, excipients or diluents

In other aspects, provided are methods for the treatment or prophylaxis of a disease or disorder of the respiratory, urinary or gastrointestinal system mediated through the muscarinic receptors comprising administering to an animal or human in need thereof a therapeutically effective amount of a compound described herein or a pharmaceutically acceptable salt, pharmaceutically acceptable solvate, enantiomer, diastereomer, polymorph or N-oxide thereof; or a pharmaceutical composition thereof.

The disease of disorder can be selected from urinary incontinence, lower urinary tract symptoms (LUTS), bronchial asthma, chronic obstructive pulmonary disease (COPD), pulmonary fibrosis, irritable bowel syndrome, obesity, diabetes or gastrointestinal hyperkinesis.

In another aspect, provided are pharmaceutical compositions comprising a therapeutically effective amount of a compound described herein or a pharmaceutically acceptable salt, pharmaceutically acceptable solvate, enantiomer, diastereomer, polymorph or N-oxide thereof; and one or more therapeutic agent selected from one or more corticosteroids, beta agonists, leukotriene antagonists, 5-lipoxygenase inhibitors, anti-histamines, antitussives, dopamine receptor antagonists, chemokine inhibitors, p38 MAP Kinase inhibitors, PDE-IV inhibitors or a mixture thereof.

In another aspect, provided are methods of preparing a compound of Formula VII comprising the steps of:
a. reacting a compound of Formula II

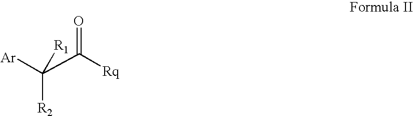

with a compound of Formula III

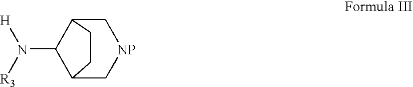

to form a compound of Formula IV,

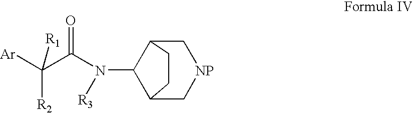

b. deprotecting the compound of Formula IV to form a compound of Formula V, and

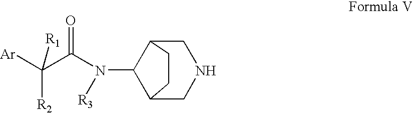

c. reacting a compound of Formula V with a compound of Formula VI

R₄-hal Formula VI
to form a compound of Formula VII,

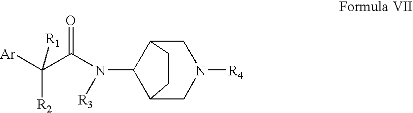

wherein
Ar can be aryl or heteroaryl;
R₁ can be hydrogen, hydroxy, alkyl, halogen or alkoxy;
R₂ can be cycloalkyl, aryl or heteroaryl;
R₃ can be hydrogen or alkyl;
R₄ can be alkyl, alkenyl, aralkyl or heteroarylalkyl;

### Rq can be hal (CI, Br or I) or —OH; and

P can be aralkyl.

In another aspect, provided are methods of preparing a compound of Formula X comprising the steps of:
a. reacting a compound of Formula II

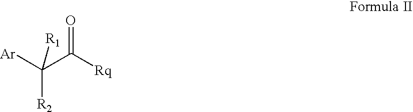

with a compound of Formula III

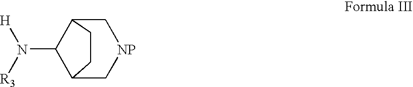

to form a compound of Formula IV,

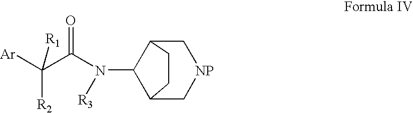

b. hydroxylating a compound of Formula IV (wherein R₁ is halogen) to form a compound of Formula VIII,

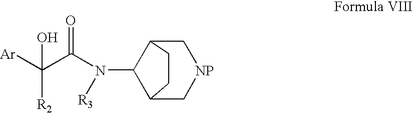

c. deprotecting a compound of Formula VIII to form a compound of Formula IX, and

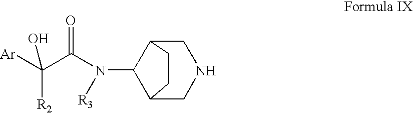

d. reacting a compound of Formula IX with a compound of Formula VI to form a compound of Formula X,

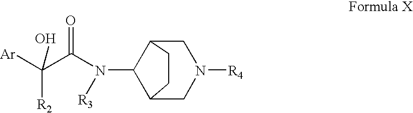

wherein
Ar can be aryl or heteroaryl;

### Rq can be hal (CI, Br or I) or —OH;

R₁ can be hydrogen, hydroxy, alkyl, halogen or alkoxy;
R₂ can be cycloalkyl, aryl or heteroaryl;
R₃ can be hydrogen or alkyl;
R₄ can be alkyl, alkenyl, aralkyl or heteroarylalkyl; and
P can be aralkyl.

### DETAILED DESCRIPTION OF THE INVENTION

In accordance with one aspect, provided are compounds having the structure of Formula I:

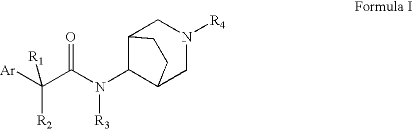

and pharmaceutically acceptable salts, pharmaceutically acceptable solvates, enantiomers, diastereomers, polymorphs or N-oxides thereof, wherein
Ar can be aryl or heteroaryl;
R₁ can be hydrogen, hydroxy, alkyl, halogen or alkoxy;
R₂ can be cycloalkyl, aryl or heteroaryl;
R₃ can be hydrogen or alkyl; and
R₄ can be alkyl, alkenyl, aralkyl or heteroarylalkyl.

In another aspect, provided are methods for the treatment or prophylaxis of a disease or disorder of the respiratory, urinary and gastrointestinal systems comprising administering to an animal or human in need thereof one or more compounds having the structure of Formula I, wherein the disease or disorder is mediated through muscarinic receptors.

In another aspect, provided are methods for the treatment or prophylaxis of a disease or disorder of the respiratory system (for example, bronchial asthma, chronic obstructive pulmonary disorders (COPD), pulmonary fibrosis, and the like); urinary system which induce urinary disorders (for example, urinary incontinence, lower urinary tract symptoms (LUTS), etc.), or gastrointestinal system (for example, irritable bowel syndrome, obesity, diabetes and gastrointestinal hyperkinesis) by administering one or more compounds described herein to an animal or human in need thereof, wherein the disease or disorder is associated with muscarinic receptors.

In another aspect, provided are processes for preparing the compounds as described above.

Compounds described herein exhibit affinity for M₃ receptors, as determined by in vitro receptor binding assay.

Compounds disclosed herein may be prepared by the reaction sequences as generally shown in Scheme I.

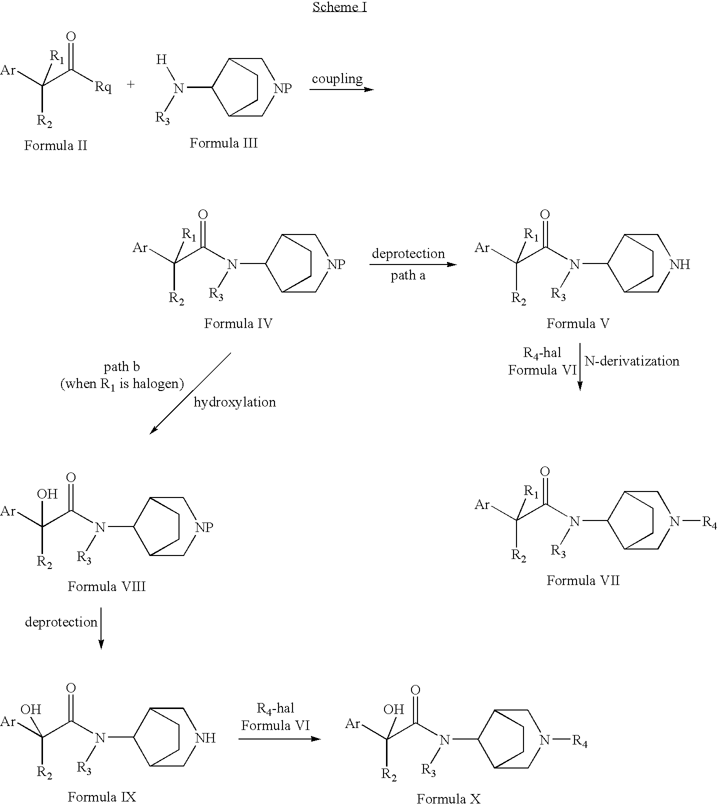

The compounds of Formula IV, V, VII, VIII, 1× and X can be prepared by following the procedure as depicted in Scheme I. Thus, a compound of Formula II (wherein Rq is hal (Cl, Br or I) or —OH; Ar, R₁ and R₂ are the same as defined earlier) can be reacted with a compound of Formula III (wherein P is aralkyl and R₃ is the same as defined earlier) to form a compound of Formula IV.

Path a: A compound of Formula IV can be deprotected to form a compound of Formula V; and a compound of Formula V can be reacted with a compound of Formula VI to form a compound of Formula VII (wherein R₄ is the same as defined earlier).

Path b: A compound of Formula IV can be hydroxylated to form a compound of Formula VIII; a compound of Formula VIII can be deprotected to form a compound of Formula IX; and a compound of Formula IX can be N-derivatized with a compound of Formula VI to form a compound of Formula X.

Compounds of Formula II (wherein Rq is hal) can be coupled with compounds of Formula III (wherein R₃ is alkyl) to form compounds of Formula IV in one or more organic solvents. Suitable organic solvents include, for example, dichloromethane, dichloroethane, chloroform, carbon tetrachloride or mixtures thereof. The coupling reaction can also be carried out in the presence of one or more bases, for example, triethylamine, pyridine, N-methylmorpholine, diisopropylethylamine or mixtures thereof.

Compounds of Formula II (wherein Rq is —OH) can be coupled with compounds of Formula III (wherein R₃ is hydrogen) to form compounds of Formula IV in one or more organic solvents (for example, dimethylformamide, chloroform, tetrahydrofuran, diethyl ether, dioxane or mixtures thereof). The coupling reaction can also be carried out in the presence of one or more bases (for example, N-methylmorpholine, triethylamine, diisopropylethylamine, pyridine or mixtures thereof) with one or more condensing agents, for example, 1-(3-dimethylaminopropyl)-3-ethylcarbodiimide hydrochloride (EDC′HCl), dicyclohexylcarbodiimide (DCC) or mixtures thereof).

Compounds of Formula IV (path a) can be deprotected to form compounds of Formula V can be carried out in one or more organic solvents (for example, ethyl acetate, methanol, ethanol, propanol, isopropyl alcohol or mixtures thereof). The deprotection reaction can also be carried out in the presence of one or more deprotecting agents (for example, palladium on carbon in presence of hydrogen gas or palladium on carbon with a source of hydrogen gas (for example, ammonium formate, cyclohexene, formic acid or mixtures thereof)).

Compounds of Formula V can be N-derivatized with compounds of Formula VI to form compounds of Formula VI in one or more organic solvents (for example, acetonitrile, dichloromethane, chloroform, carbon tetrachloride or mixtures thereof). The N-derivatization can also be carried out in the presence of one or more bases (for example, potassium carbonate, sodium carbonate, sodium bicarbonate or mixtures thereof).

Compounds of Formula IV (path b) can be hydroxylated to form compounds of Formula VIII in one or more organic solvents (for example, dioxane, diethyl ether, tetrahydrofuran) in the presence of an acid (or example, hydrochloric acid). The hydroxylation can be carried out under reflux conditions.

Compounds of Formula VIII can be deprotected to form compounds of Formula IX in one or more organic solvents (for example, ethyl acetate, methanol, ethanol, propanol, isopropyl alcohol or mixtures thereof). The deprotection can also be carried out in the presence of one or more deprotecting agents (for example, palladium on carbon in presence of hydrogen gas or palladium on carbon with a source of hydrogen gas (for example, ammonium formate, cyclohexene, formic acid or mixtures thereof)).

Compounds of Formula IX can be N-derivatized with compounds of Formula VI to form compounds of Formula X in one or more organic solvents (for example, acetonitrile, dichloromethane, chloroform, carbon tetrachloride or mixtures thereof). The N-derivatization can also be carried out in the presence of one or more bases (for example, potassium carbonate, sodium carbonate, sodium bicarbonate or mixtures thereof).

Compounds prepared following Scheme I, path a include, for example:
N-(3-benzyl-3-azabicyclo[3.2.1]oct-8-yl)-N-methyl-2,2-diphenylacetamide (Compound No. 1),
N-3-azabicyclo[3.2.1]oct-8-yl-N-methyl-2,2-diphenylacetamide (Compound No. 2),
N-(3-benzyl-3-azabicyclo[3.2.1]oct-8-yl)-N-methyl-2,2-diphenylpropanamide (Compound No. 4),
(Exo or Endo) N-(3-benzyl-3-azabicyclo[3.2.1]oct-8-yl)-2-cyclopentyl-2-hydroxy-2-(4-methylphenyl)acetamide (Compound No. 5),
(Exo or Endo) N-(3-benzyl-3-azabicyclo[3.2.1]oct-8-yl)-2-cyclopentyl-2-hydroxy-2-(4-methylphenyl)acetamide (Compound No. 6),
N-(3-benzyl-3-azabicyclo[3.2.1]oct-8-yl)-2-hydroxy-2-(4-methoxyphenyl)-2-cyclopentylacetamide (Compound No. 7),
N-(3-benzyl-3-azabicyclo[3.2.1]oct-8-yl)-2-cyclopentyl-2-hydroxy-2-(4-methoxyphenyl)acetamide (Compound No. 8),
N-3-azabicyclo[3.2.1]oct-8-yl-2-cyclopentyl-2-hydroxy-2-(4-methoxyphenyl)acetamide (Compound No. 9),
N-(3-benzyl-3-azabicyclo[3.2.1]oct-8-yl)-2-chloro-N-methyl-2,2-diphenylacetamide (Compound No. 10),
N-3-azabicyclo[3.2.1]oct-8-yl-2-cyclopentyl-2-hydroxy-2-(4-methylphenyl)acetamide (Compound No. 11),
(Exo or Endo) N-(3-benzyl-3-azabicyclo[3.2.1]oct-8-yl)-2,2-diphenylacetamide (Compound No. 13),
(Exo or Endo) N-(3-benzyl-3-azabicyclo[3.2.1]oct-8-yl)-2,2-diphenylacetamide (Compound No. 14),
N-(3-benzyl-3-azabicyclo[3.2.1]oct-8-yl)-2-hydroxy-2-phenyl-2-(2-thienyl)acetamide (Compound No. 17),

Compounds prepared following Scheme I, path b include, for example:
N-(3-benzyl-3-azabicyclo[3.2.1]oct-8-yl)-2-hydroxy-N-methyl-2,2-diphenylacetamide (Compound No. 3),
N-3-azabicyclo[3.2.1]oct-8-yl-2-hydroxy-N-methyl-2,2-diphenylacetamide (Compound No. 12),
2-hydroxy-N-methyl-N-[3-(4-methylpent-3-en-1-yl)-3-azabicyclo[3.2.1]oct-8-yl]-2,2-diphenylacetamide (Compound No. 15),
N-{3-[2-(1,3-benzodioxol-5-yl)ethyl]-3-azabicyclo[3.2.1]oct-8-yl}-2-hydroxy-N-methyl-2,2-diphenylacetamide (Compound No. 16).

In the above schemes, where specific reagents (for example, bases, condensing agents, protecting groups, deprotecting agents, solvents, catalysts, temperatures, etc.) are described, other reagents (for example, bases, condensing agents, protecting groups, deprotecting agents, solvents, catalysts, temperatures, etc.) known to one of ordinary skill in the art may be used. Similarly, reaction conditions (for example, temperature and duration) may be adjusted according to the desired needs.

Suitable salts of compounds described herein can be prepared to, for example, solubilize compounds in aqueous medium for biological evaluations, as well as to make them compatible with various dosage forms and to aid in the bioavailability of the compounds. Examples of such salts include pharmacologically acceptable salts, for example, inorganic acid salts (for example, hydrochloride, hydrobromide, sulphate, nitrate and phosphate); and organic acid salts (for example, acetate, tartarate, citrate, fumarate, maleate, tolounesulphonate and methanesulphonate). When carboxyl groups are present as substituents in the compounds described herein, they may be present in the form of an alkaline or alkali metal salt (for example, sodium, potassium, calcium, magnesium, and the like). Such salts may be prepared by various techniques, such as treating compounds with an equivalent amount of one or more inorganic or organic acids or bases in a suitable solvent.

The compounds described herein can be formulated as their enantiomers, diastereomers, N-oxides, polymorphs, solvates and pharmaceutically acceptable salts, as well as metabolites having the same type of activity. Pharmaceutical compositions comprising one or more compounds described herein or metabolites, enantiomers, diastereomers, N-oxides, polymorphs, solvates or pharmaceutically acceptable salts thereof, in combination with one or more pharmaceutically acceptable carriers, excipients or diluents can also be produced.

Where desired, the compounds described herein and/or their pharmaceutically acceptable salts, pharmaceutically acceptable solvates, stereoisomers, tautomers, racemates, prodrugs, metabolites, polymorphs or N-oxides may be used in combination with one or more other therapeutic agents. Examples of other therapeutic agents include, but are not limited to, one or more corticosteroids, beta agonists, leukotriene antagonists, 5-lipoxygenase inhibitors, anti-histamines, antitussives, dopamine receptor antagonists, chemokine inhibitors, p38 MAP Kinase inhibitors, PDE-IV inhibitors or any combination thereof.

Compounds described herein may be administered to a patient (for example, animal or human) for treatment by any route of administration. Suitable routes of administration include, for example, oral or parenteral routes. Pharmaceutical compositions described herein can be produced and administered in dosage units, each unit containing a certain amount of at least one compound described herein and/or at least one physiologically acceptable addition salt thereof. Dosage units may be varied over extremely wide limits, as the compounds can be effective at low dosage levels and relatively free of toxicity. The compounds may be administered in low micromolar concentration, which is therapeutically effective, and the dosage may be increased as desired up to a maximum dosage tolerated by the patients.

Pharmaceutical compositions for inhalation or insufflation include solutions and suspensions in one or more pharmaceutically acceptable aqueous or organic solvents or mixtures thereof, and powders. Liquid or solid compositions may contain one or more suitable pharmaceutically acceptable excipients. Pharmaceutical compositions can be administered by the nasal respiratory route for local or systemic effect. Compositions can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered nasally from devices, which deliver the formulation in an appropriate manner.

Alternatively, compositions can be administered orally, rectally, parenterally (intravenously, intramuscularly or subcutaneously), intratracheal, intraarterial, intraperitoneal, intrathecal, intraventricular, intraurethral, intrasternal, intracranial, subcutaneous, intranasal, intracisternally, intravaginally, intraperitoneally or topically.

Solid dosage forms for oral administration may be presented in discrete units, for example, capsules, cachets, lozenges, tablets, pills, powders, dragees or granules, each containing a predetermined amount of the active compound. In such solid dosage forms, one or more active compounds can be admixed with at least one inert customary excipient (or carrier) such as sodium citrate or dicalcium phosphate or (a) fillers or extenders, as for example, starches, lactose, sucrose, glucose, mannitol and silicic acid, (b) binders, as for example, carboxymethylcellulose, alginates, gelatin, polyvinylpyrrolidone, sucrose and acacia, (c) humectants, as for example, glycerol, (d) disintegrating agents, as for example, agar-agar, calcium carbonate, potato or tapioca starch, alginic acid, certain complex silicates and sodium carbonate, (e) solution retarders, as for example paraffin, (f) absorption accelerators, as for example, quaternary ammonium compounds, (g) wetting agents, as for example, cetyl alcohol and glycerol monostearate, (h) adsorbents, as for example, kaolin and bentonite, and (i) lubricants, as for example, talc, calcium stearate, magnesium stearate, solid polyethylene glycols, sodium lauryl sulfate or mixtures thereof. In the case of capsules, tablets and pills, the dosage forms may also comprise buffering agents.

Solid compositions of a similar type may also be employed as fillers in soft and hard-filled gelatin capsules using such excipients as lactose or milk sugar as well as high molecular weight polyethylene glycols, and the like.

Solid dosage forms can include coatings and shells, such as enteric coatings and other coatings or shells well known in this art. Solid dosage forms may contain opacifying agents and can be of such composition that facilitates delayed release of one or more active compounds in a certain part of the intestinal tract. Examples of embedding compositions which can be used include polymeric substances and waxes.

Active compounds can also be micro-encapsulated, if appropriate, with one or more of the above mentioned excipients or any other excipient known in the art.

Liquid dosage forms for oral administration include pharmaceutically acceptable emulsions, solutions, suspensions, syrups and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil and sesame oil, glycerol, tetrahydrofurfuryl alcohol, polyethylene glycols and fatty acid esters of sorbitan or mixtures of these substances, and the like.

Pharmaceutical compositions can also include adjuvants, for example, wetting agents, emulsifying and suspending agents, sweetening, flavoring and perfuming agents, colorants or dyes.

Suspensions may contain suspending agents, as for example, ethoxylated isostearyl alcohols, polyoxyethylene sorbitol and sorbitan esters, microcrystalline cellulose, aluminium metahydroxide, bentonite, agar-agar and tragacanth, or mixtures of these substances, and the like.

Dosage forms for topical administration include powder, spray, inhalant, ointment, creams, salve, jelly, lotion, paste, gel, aerosol, or oil. Active compound(s) can be admixed under sterile conditions with one or more pharmaceutically acceptable carrier and option preservatives, buffers or propellants as may be required. Ophthalmic formulations, eye ointments, powders and solutions are also encompassed.

Pharmaceutical compositions suitable for parenteral injection include pharmaceutically acceptable sterile aqueous or nonaqueous solutions, dispersions, suspensions or emulsions and sterile powders for reconstitution into sterile injectable solutions or dispersions. Such pharmaceutical compositions may contain anti-oxidants, buffers, bacteriostats and solutes, which render the compositions isotonic with the blood of the intended recipient. Aqueous and non-aqueous sterile suspensions may include suspending agents and thickening agents. The pharmaceutical compositions may be presented in unit-dose or multi-dose containers, for example sealed ampoules and vials, and may be stored in a freeze-dried or lyophilized condition requiring only the addition of the sterile liquid carrier, for example, saline or water-for-injection immediately prior to use. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

Pharmaceutical compositions may also contain adjuvants, for example, preserving, wetting, emulsifying, and dispensing agents. Pharmaceutical compositions may also comprise one or more antibacterial and antifungal agents, for example, parabens, chlorobutanol, phenol, sorbic acid, and the like. Pharmaceutical compositions can also include isotonic agents, for example, sugars, sodium chloride and the like. Prolonged absorption of the injectable pharmaceutical form can be brought about by the use of agents delaying absorption, for example, aluminum monosterate and gelatin.

Suppositories for rectal administration of the compound of Formula I can be prepared by mixing the drug with a suitable nonirritating excipient such as cocoa butter and polyethylene glycols or a suppository wax, which are solid at ambient temperatures but liquid at body temperature and which therefore melt in the rectum or vaginal cavity and release the drug.

For more effective distribution, compounds described herein can be incorporated into slow release or targeted delivery systems, for example, polymer matrices, liposomes, and microspheres. They may be sterilized, for example, by filtration through a bacteria-retaining filter, or by incorporating sterilizing agents in the form of sterile solid compositions, which can be dissolved in sterile water, or some other sterile injectable medium immediately before use.

Actual dosage levels of active compounds in pharmaceutical compositions and spacing of individual dosages may be varied to obtain a desired therapeutic response for a particular composition and method of administration. Specific dosage levels for any particular patient can depend upon a variety of factors including, for example, the particular compound chosen, body weight, general health, sex, diet, route of administration, the desired duration of treatment, rates of absorption and excretion, combination with other drugs and the severity of the particular disease being treated and is ultimately at the discretion of the physician.

Pharmaceutical compositions described herein can be produced and administered in dosage units, each unit containing a certain amount of at least one compound described herein and/or at least one physiologically acceptable addition salt thereof. The dosage may be varied over extremely wide limits as the compounds are effective at low dosage levels and relatively free of toxicity. The compounds may be administered in the low micromolar concentration, which is therapeutically effective, and the dosage may be increased as desired up to the maximum dosage tolerated by the patient.

While the present invention has been described in terms of its specific embodiments, certain modifications and equivalents will be apparent to those skilled in the art and are included within the scope of the present invention. The examples are provided to illustrate particular aspects of the disclosure and do not limit the scope of the present invention as defined by the claims.

### EXAMPLES

Solvents used herein, such as acetone, methanol, pyridine, ether, tetrahydrofuran, hexanes, and dichloromethane, were dried using various drying reagents according to procedures described in the literature. IR spectra were recorded as nujol mulls or a thin neat film on a Perkin Elmer Paragon instrument, Nuclear Magnetic Resonance (NMR) were recorded on a Varian XL-300 MHz or Bruker 400 MHz instrument using tetramethylsilane as an internal standard.

### Example A

### Synthesis of Chloro(diphenyl)acetyl Chloride

Thionyl chloride (1.3 mL, 17.54 mmol) was added to a solution of hydroxy(diphenyl)acetic acid (1 g, 4.38 mmol) in dichloromethane (2 mL) and the mixture was refluxed for 2 hours. Excess thionyl chloride was distilled off to yield the title compound.

### Example B

### Synthesis of 3-benzyl-N-methyl-3-azabicyclo[3.2.1]octan-8-amine

A solution of (3-benzyl)-3-((azabicyclo[3.2.1]octanone and methyl amine hydrochloride in methanol (10 mL) was added under nitrogen atmosphere to a solution of sodium cyanoborohydride (0.146 g, 2.32 mmol) and zinc chloride (0.158 g, 1.16 mmol) in methanol (15 mL). The mixture was stirred at room temperature for 3 hours and subsequently quenched by adding sodium hydroxide (6N). The mixture was filtered through a celite pad and the filtrate was concentrated under reduced pressure. The residue thus obtained was diluted with water and extracted with ethyl acetate. The organic layer was dried over anhydrous sodium sulphate, filtered and concentrated under reduced pressure. The residue was dissolved in dilute hydrochloric acid solution and extracted with dichloromethane. The aqueous layer was basified with sodium hydroxide solution and extracted with ethyl acetate. The organic layer was washed with brine, dried over anhydrous sodium sulphate and concentrated under reduced pressure to yield the title compound. Yield: 400 mg.

¹H NMR (CDCl₃): δ 7.19-7.34 (m, 5H), 3.51 (s, 2H), 2.69 (s, 2H), 2.69-2.67 (t, 1H), 2.51-2.37 (m, 7H), 2.08-2.03 (bs, 2H), 1.86-1.84 (m, 2H).

Scheme L path a:

### Example 1

### Synthesis of N-(3-benzyl-3-azabicyclo[3.2.1]oct-8-yl)-N-methyl-2,2-diphenylacetamide (Compound No. 1)

A solution of diphenyl acetyl chloride (0.22 g, 0.95 mmol) in dichloromethane (5 mL) was added to a solution of the 3-benzyl-N-methyl-3-azabicyclo[3.2.1]octan-8-amine (0.219 g, 0.95 mmol) and triethylamine (0.43 g, 4.33 mmol) in dichloromethane (2 mL) and the mixture was stirred at room temperature for 4 hours. The reaction mixture was concentrated under reduced pressure and the residue thus obtained was washed sodium bicarbonate and extracted with dichloromethane. The organic layer was dried and concentrated under reduced pressure. The residue thus obtained was purified by column chromatography using 15% ethyl acetate in hexane as eluent to yield the title compound. Yield: 0.17 g.

¹H NMR (CDCl₃): δ 7.35-7.26 (m, 15H), 5.26 (s, 1H), 3.42 (s, 3H), 3.03 (s, 3H), 2.74 (s, 2H), 2.46-2.42 (m, 2H), 2.20-2.17 (m, 2H), 1.82-1.80 (m, 2H), 1.66-1.63 (m, 2H). Mass (m/z): 425 (M⁺+1).

The following compounds were prepared similarly using the appropriate corresponding reagents:

### N-(3-benzyl-3-azabicyclo[3.2.1]oct-8-yl)-N-methyl-2,2-diphenylpropanamide (Compound No. 4)

¹H NMR (CDCl₃): δ 7.40-7.22 (m, 15H), 3.46 (s, 2H), 3.42-3.40 (m, 1H), 2.75 (bs, 2H), 2.45-2.41 (m, 4H), 2.13 (d, 1H), 1.91 (s, 3H), 1.87-1.82 (m, 2H), 1.68-1.65 (m, 2H), 1.42-1.41 (m, 2H).

Mass (m/z): 439 (M⁺+1).

### N-(3-benzyl-3-azabicyclo[3.2.1]oct-8-yl)-2-chloro-N-methyl-2,2-diphenylacetamide (Compound No. 10)

### Example 2

### Synthesis of N-3-azabicyclo[3.2.1]oct-8-yl-N-methyl-2,2-diphenylacetamide (Compound No. 2)

A solution of Compound No. 1 (0.29 g), palladium on carbon (10%, 100 mg) and ammonium formate (0.215 g, 3.42 mmol) in methanol (200 mL) was refluxed for 1 hour. The reaction mixture was filtered through a celite pad and washed with methanol. The filtrate was concentrated under reduced pressure and the residue thus obtained was diluted with water. The mixture was basified with sodium hydroxide solution and extracted with ethyl acetate. The organic layer was washed with brine, dried over anhydrous sodium sulphate and concentrated under reduced pressure to yield the title compound. Yield: 140 mg.

¹H NMR (CDCl₃): δ7.36-7.22 (m, 10H), 5.26 (s, 1H), 3.49 (m, 1H), 3.06 (m, 4H), 2.83-2.70 (m, 4H), 2.57-2.10 (m, 6H).

Mass (m/z): 335 (M⁺+1).

### Example 3

### Synthesis of (Exo or Endo)N-(3-benzyl-3-azabicyclo[3.2.1]oct-8-yl)-2-cyclopentyl-2-hydroxy-2-(4-methylphenyl)acetamide (Compound No. 5) and (Exo or Endo) N-(3-benzyl-3-azabicyclo[3.2.1]oct-8-yl)-2-cyclopentyl-2-hydroxy-2-(4-methylphenyl)acetamide (Compound No. 6)

To a solution of hydroxy(diphenyl)acetic acid (324 mg) and 3-benzyl-3-azabicyclo[3.2.1]octan-8-amine (300 mg) in dimethylformamide (10 mL) were added hydroxybenzotriazole (187 mg) and N-methylmorpholine (0.30 mL) at 0° C. The mixture was stirred at 0° C. for 1 hour followed by the addition of 1-(3-dimethylaminopropyl)-3-ethylcarbodiimide hydrochloride (266 mg). The reaction mixture was further stirred at 0° C. for 1 hour, then at room temperature overnight followed by adding sodium bicarbonate solution to quench the reaction. The mixture was extracted with ethylacetate, the ethylacetate layer was washed with water and brine, dried over anhydrous sodium sulphate and concentrated under reduced pressure. The residue thus obtained was purified by column chromatography using 8% ethyl acetate in hexane as eluent to yield the title Compound No. 5 (0.16 g) and using 8% ethyl acetate in hexane as eluent to yield the title Compound No. 6. Yield: 0.1 g.

The following compounds were prepared similarly using the appropriate corresponding reagents:

### N-(3-benzyl-3-azabicyclo[3.2.1]oct-8-yl)-2-hydroxy-2-(4-methoxyphenyl)-2-phenylacetamide (Compound No. 7)

¹H NMR (CDCl₃₎: δ 7.49 (2H, d), 7.29 (5H, m), 6.87 (2H, d), 3.79 (3H, s), 3.66 (1H, d), 3.45 (2H, s), 2.93 (1H, q), 2.61 (2H, m), 2.1-1.2 (16H, m).

Mass (m/z): 449 (M⁺+1).

### N-(3-benzyl-3-azabicyclo[3.2.1]oct-8-yl)-2-cyclopentyl-2-hydroxy-2-(4-methoxyphenyl)acetamide (Compound No. 8)

¹H NMR (CDCl₃₎: δ 7.50 (2H, d), 7.28 (5H, m), 6.88 (2H, d), 3.81 (3H, s), 3.71 (1H, m), 3.44 (2H, s), 2.92 (2H, m), 2.0-1.2 (16H, m).

Mass (m/z): 499 (M⁺+1).

### (Exo or Endo) N-(3-benzyl-3-azabicyclo[3.2.1]oct-8-yl)-2,2-diphenylacetamide (Compound No. 13)

¹H NMR (CDCl₃₎: δ 7.35-7.21 (15H, m), 4.90 (1H, s), 3.81 (1H, d), 3.46 (2H, s), 2.61 (2H, d), 2.22 (2H, d), 2.09 (2H, m), 1.57 (4H, m).

Mass (m/z): 411 (M⁺+1).

### (Exo or Endo) N-(3-benzyl-3-azabicyclo[3.2.1]oct-8-yl)-2,2-diphenylacetamide (Compound No. 14)

¹H NMR (CDCl₃₎: δ 7.40-7.22 (15H, m), 5.02 (1H, s), 3.96 (1H, m), 3.27 (2H, s), 2.42 (2H, d), 2.08 (2H, s), 1.89-1.69 (6H, m).

Mass (m/z): 411 (M⁺+1).

### N-(3-benzyl-3-azabicyclo[3.2.1]oct-8-yl)-2-hydroxy-2-phenyl-2-(2-thienyl)acetamide (Compound No. 17)

¹H NMR (CDCl₃₎: δ 7.53-7.25 (13H, m), 3.91 (1H, m), 3.30 (2H, s), 2.48-1.80 (10H, m).

Mass (m/z): 433 (M⁺+1).

### Example 4

### Synthesis of N-3-azabicyclo[3.2.1]oct-8-yl-2-cyclopentyl-2-hydroxy-2-(4-methoxyphenyl)acetamide (Compound No. 9)

The title compound was prepared following the procedure as described in Example 2, by using Compound No. 8 in place of Compound No. 1.

¹H NMR (CDCl₃₎: δ 7.56 (2H, d), 6.89 (2H, d), 3.84 (4H, s), 2.9-1.6 (20H, m).

Mass (m/z): 359 (M⁺+1).

The following compounds were prepared similarly using the appropriate corresponding reagents:

### N-3-azabicyclo[3.2.1]oct-8-yl-2-cyclopentyl-2-hydroxy-2-(4-methylphenyl)acetamide (Compound No. 11)

¹H NMR (CDCl₃₎: δ 7.53 (2H, d), 7.16 (2H, d), 3.86 (1H, m), 3.0-2.53 (3H, m), 2.48-1.6 (16H, m).

Mass (m/z): 343 (M⁺+1).

Scheme I, path b:

### Example 5

### Synthesis of N-(3-benzyl-3-azabicyclo[3.2.1]oct-8-yl)-2-hydroxy-N-methyl-2,2-diphenylacetamide (Compound No. 3)

A solution of Compound No. 10 (0.35 g) in dioxane (30 mL) and hydrochloric acid (15 mL) was refluxed for 1 hour. The mixture was concentrated under reduced pressure and the residue thus obtained was basified with sodium hydroxide and extracted with ethyl acetate. The organic layer was washed with water and brine, dried over anhydrous sodium sulphate, filtered and concentrated under reduced pressure. The residue thus obtained was purified by column chromatography to yield the title compound. Yield: 210 mg.

¹H NMR (CDCl₃): 7.45-7.19 (m, 15H), 3.51 (t, 1H), 3.37 (s, 2H), 2.76 (bs, 1H), 2.59 (s, 2H), 2.45-2.40 (m, 2H), 1.99 (m, 1H), 1.84 (m, 2H), 1.67 (m, 3H), 1.26 (s, 2H).

Mass (m/z): 441 (M⁺+1).

IR: 1640 cm⁻¹

### Example 6

### Synthesis of N-3-azabicyclo[3.2.1]oct-8-yl-2-hydroxy-N-methyl-2,2-diphenylacetamide (Compound No. 12)

The title compound was prepared following the procedure as described in Example 2 by using Compound No. 3 in place of Compound No. 1.

¹H NMR (CDCl₃₎: δ 7.42-7.33 (m, 10H), 3.62 (t, 1H), 2.83-2.55 (m, 8H), 2.05-1.68 (m, 6H).

### Example 7

### Synthesis of 2-hydroxy-N-methyl-N-[3-(4-methylpent-3-en-1-yl)-3-azabicyclo[3.2.1]oct-8-yl]-2,2-diphenylacetamide (Compound No. 15)

Potassium carbonate (49 mg), potassium iodide (29.4 mg) and 5-bromo-2-methyl-2-pentene (35.3 μL) was added to a solution of Compound No. 12 (62 mg) in acetonitrile (3 mL). The mixture was stirred at 80° C. for 5 hours and subsequently at room temperature overnight. The mixture was concentrated under reduced pressure and the residue thus obtained was partitioned between dichloromethane and water. The separated organic layer was washed with water and brine, dried over anhydrous sodium sulphate and concentrated under reduced pressure. The residue thus obtained was purified by preparative column chromatography to yield the title compound. Yield: 27 mg

¹H NMR (CDCl₃₎: δ 7.43-7.33 (10H, m), 5.07-5.04 (1H, m), 3.65-3.47 (1H, m), 3.15-2.05 (13H, m), 21.78-1.73 (6H, m), 1.33-1.1 (4H, m).

Mass (m/z): 433 (M⁺+1).

The following compound was prepared similarly using the appropriate corresponding reagents:

### N-{3-[2-(1,3-benzodioxol-5-yl)ethyl]-3-azabicyclo[3.2.1]oct-8-yl}-2-hydroxy-N-methyl-2,2-diphenylacetamide (Compound No. 16)

¹H NMR (CDCl₃₎: δ 7.42-7.32 (10H, m), 6.78-6.66 (3H, m), 5.95 (2H, s), 3.49-3.47 (2H, m), 2.89-2.52 (11H, m), 2.18-2.05 (4H, m).

Mass (m/z): 499 (M⁺+1).

### Example 8

### Biological Activity

### In-Vitro Experiments

### Radioligand Binding Assays:

The affinity of test compounds for M₂ and M₃ muscarinic receptor subtypes was determined by [³H]-N-methylscopolamine binding studies using rat heart and submandibular gland respectively as described by Moriya et ah, (Life Sci., 1999, 64(25):2351-2358) with minor modifications. In competition binding studies, specific binding of [3H]NMS was also determined using membranes from Chinese hamster ovary (CHO) cells expressing cloned human M₁, M₂, M₃, M₄ and M₅ receptors. Selectivities were calculated from the Ki values obtained on these human cloned membranes.

Membrane preparation:

### (a) Rat Tissues

Submandibular glands and heart were isolated and placed in ice-cold homogenising buffer (HEPES 20 mM, 10 mM EDTA, pH 7.4) immediately after sacrifice. The tissues were homogenised in ten volumes of homogenising buffer, the homogenate was filtered through two layers of wet gauze and the filtrate was centrifuged at 500 g for 10 minutes. The supernatant was subsequently centrifuged at 40,000 g for 20 minutes. The pellet thus obtained was resuspended in assay buffer (HEPES 20 mM, EDTA 5 mM, pH 7.4) and were stored at −70° C. until the time of assay.

### (b) CHO Cells Expressing Human Recombinant Receptors

The cell pellets were homogenised for 30 seconds at 12,000 to 14,000 rpm, with intermittent gaps of 10-15 seconds in ice-cold homogenising buffer (20 mM HEPES, 10 mM EDTA, pH 7.4). The homogenate was then centrifuged at 40,000 g for 20 min at 4° C. The pellet thus obtained was resuspended in homogenising buffer containing 10% sucrose and was stored at −70° C. until the time of assay.

Ligand binding assay: The compounds were dissolved and diluted in DMSO. The membrane homogenates (150-250 μg protein) were incubated in 250 μL of assay volume (HEPES 20 mM, pH 7.4) at 24-25° C. for 3 hours. Non-specific binding was determined in the presence of 1 μM atropine. The incubation was terminated by vacuum filtration over GF/B fiber filters (Wallac). The filters were then washed with ice-cold 50 mM Tris HCl buffer (pH 7.4). The filter mats were dried and bound radioactivity retained on filters was counted. The IC₅₀ & K_{d} were estimated by using the non-linear curve fitting program using G Pad Prism software. The value of inhibition constant K was calculated from competitive binding studies by using Cheng & Prusoff equation (Biochem Pharmacol, 1973, 22:3099-3108), Ki=IC₅₀/(1+L/Kd), where L is the concentration of [³H]NMS used in the particular experiment, pki is −log [Ki].

Tested compounds showed pKi values for M₃ from about 12 to about 1000 nM, from about 12 to about 361 nM and even from about 12 to about 97 nM.

Tested compounds showed pKi values for M₂ from about 10 to about 1000 nM, from about 10 to about 358 nM and even from about 10 to about 290 nM.

### Functional Experiments Using Isolated Rat Bladder:

### Methodology:

Animals are euthanized by overdose of thiopentone and whole bladder is isolated and removed rapidly and placed in ice cold Tyrode buffer with the following composition (mMol/L) NaCl 137; KCl 2.7; CaCl₂ 1.8; MgCl₂ 0.1; NaHCO₃ 11.9; NaH2PO₄ 0.4; Glucose 5.55 and continuously gassed with 95% O₂ and 5% CO₂.

The bladder is cut into longitudinal strips (3 mm wide and 5-6 mm long) and mounted in 10 mL organ baths at 30° C., with one end connected to the base of the tissue holder and the other end connected through a force displacement transducer. Each tissue is maintained at a constant basal tension of 1 g and allowed to equilibrate for 1.5 hours during which the Tyrode buffer is changed every 15-20 minutes. At the end of equilibration period the stabilization of the tissue contractile response is assessed with 1 μmol/L of Carbachol until a reproducible response is obtained. Subsequently a cumulative concentration response curve to carbachol (10⁻⁹ mol/L to 3×10⁻⁴ mol/L) is obtained. After several washes, once the baseline is achieved, cumulative concentration response curve is obtained in presence of NCE (NCE added 20 minutes prior to the second cumulative response curve.

The contractile results are expressed as % of control E max. ED50 values are calculated by fitting a non-linear regression curve (Graph Pad Prism). pKb values are calculated by the formula pKb=−log [(molar concentration of antagonist/(dose ratio−1))] where the dose ratio= ED50 in the presence of antagonist/ED50 in the absence of antagonist.

In-Vitro Functional Assay to Evaluate Efficacy of MRA on Guinea Pig & Rat Trachea Animals and anaesthesia

Guinea pigs (300-900 g) are procured, their trachea removed under an overdose of anesthesia (sodium pentobarbital, ˜300 mg/kg i.p) and immediately kept in an ice-cold Krebs Henseleit buffer comprising (mM): NaCl, 118; KCl 4.7; CaCl₂, 2.5; MgSO₄, 1.2; NaHCO₃, 25; KH2PO₄, 1.2, glucose 11.1.

### Trachea Experiments:

Tissue is cleaned off adherent fascia and cut into seven-eight strips of equal size (with approximately 4-5 tracheal rings in each strip). The trachea is opened along the mid-dorsal surface with the smooth muscle band intact and a series of transverse cuts are made from alternate sides so that they do not transect the preparation completely. Opposite ends of the cut rings are tied with thread. The tissue is mounted in isolated tissue baths containing 10 mL Krebs Henseleit buffer maintained at 37° C. and the tissue baths are bubbled with carbogen (95% oxygen and 5% carbon dioxide) at a basal tension of 1 g. The buffer is changed 3-4 times for about an hour. The tissues are equilibrated for 1 hour for stabilization. After 1 hour, the tissue is contacted with 60 mM KCl, which is repeated after every 2-3 washes until two similar consecutive responses are obtained. After stabilization, a carbachol concentration-response curve on all the tissues is plotted. The tissues are washed until a baseline is obtained. Thereafter, each tissue is incubated with different concentrations of MRA/Standard/Vehicle for 20 minutes followed by plotting a second cumulative dose response curve to carbachol. The contractile response of tissues is recorded either on Powerlab system or on Grass polygraph (Model 7). The responses to carbachol is standardized as a percentage of the maximum carbachol response of the control CRC. The carbachol EC₅₀ values are determined in the presence and absence of an inhibitor using graph pad prism. pK_{B} values are calculated, an index of functional antagonism from EC₅₀ data using the following relationship:

−log [antagonist(M)/(EC₅₀antagonist/EC₅₀control)−1]

The data is expressed as a mean±s.e.m for n observations. In tissues where Eₘₐₓ attained is less than 50%, pK_{B} is calculated by Kenakin's double reciprocal plot.

All drugs and chemicals used in the study are of AR grade. Carbachol is procured from Sigma Chemicals, U.S.A. Stock solutions of Standard/NCEs are prepared in DMSO. Subsequent dilutions are prepared from the stock in MilliQ water.

In-Vitro Functional Assay to Evaluate Efficacy of “Mra” in Combination with “PDE-IV Inhibitors”

### Animals and Anaesthesia:

Guinea pigs (400-600 g) are procured, their trachea is removed under anesthesia (sodium pentobarbital, 300 mg/kg i.p) and immediately keep in ice-cold Krebs Henseleit buffer. Indomethacin (10 uM) is present throughout the KH buffer to prevent the formation of bronchoactive prostanoids.

### Trachea Experiments:

The tissue is cleaned off adherent fascia and cut into strips of equal size (with approx. 4-5 tracheal rings in each strip). The epithelium is removed by careful rubbing, minimizing damage to the smooth muscle. The trachea is opened along the mid-dorsal surface with the smooth muscle band intact and a series of transverse cuts is made from alternate sides so that they do not transect the preparation completely. Opposite ends of the cut rings are tied with thread. The tissue is mounted in isolated tissue baths containing 10 mL Krebs Henseleit buffer maintained at 37° C. and the tissue bath is bubbled with carbogen at a basal tension of 1 g. The buffer is changed 4-5 times for about an hour. The tissue is equilibrated for 1 hour for stabilization. After 1 hour, the tissue is contacted with 1 μM Carbachol, which is repeated after every 2-3 washes until two similar consecutive responses are obtained. After stabilization, the tissues are washed for 30 minutes followed by incubation with suboptimal dose of MRA/Vehicle for 20 minutes prior to contraction of the tissues with 1 uM carbachol and subsequently assess the relaxant activity of the PDE-IV inhibitor [10⁻⁹ M to 10⁻⁴ M] on the stabilized developed tension/response. The contractile response of tissues is recorded either on Powerlab data acquisition system or on Grass polygraph (Model 7). The relaxation is expressed as a percentage of maximum carbachol response. The data is expressed as a mean±s.e. mean for n observations. The EC₅₀ is calculated as the concentration producing 50% of the maximum relaxation to 1 uM carbachol. Percent relaxation is compared between the treated and control tissues using non-parametric unpaired t-test. A p value of < 0.05 is considered to be statistically significant.

### In-Vivo Experiments

In-Vivo Assay to Evaluate Efficacy of MRA Inhibitors

Male Guinea pig are anesthetized with urethane (1.5 g/kg, i.p.). The trachea is cannulated along with the jugular vein (for carbachol challenge) and specimens are placed in the Plethysmograph-Box (PLY 3114 model; Buxco Electronics, Sharon, USA.). Respiratory parameters are recorded using Pulmonary Mechanics Analyser, Biosystems XA software (Buxco Electronics, USA), which calculated lung resistance (R₁) on a breath-by-breath basis. Bronchoconstriction is induced by injections of Carbachol (10 μg/kg) delivered into the jugular vein. Increase in R_{L} over a period of 5 minutes post carbachol challenge is recorded in presence or absence of MRA or vehicle at 2 hours and 12 hours post treatment and expressed as % increase in R_{L} from basal:

$$\% Inhibition = \frac{{R}_{L vehicle} - {R}_{L test}}{{R}_{L vehicle}} \times 100$$

R_{L vehicle} % increase in lung resistance from basal in vehicle treated
R_{L test} % increase in lung resistance from basal at a given dose of test
In-Vivo Assay to Evaluate Efficacy of MRA in Combination with PDE-IV Inhibitors

### Drug Treatment:

MRA (1 μg/kg to 1 mg/kg) and PDE-IV inhibitor (1 μg/kg to 1 mg/kg) are instilled intratracheally under anesthesia either alone or in combination.

### Method:

Male wistar rats weighing 200±20 gm are used in the study. Rats have free access to food and water. On the day of experiment, animals are exposed to lipopolysaccharide (LPS, 100 μg/mL) for 40 minutes. One group of vehicle treated rats is exposed to phosphate buffered saline (PBS) for 40 minutes. Two hours after LPS/PBS exposure, animals are placed inside a whole body plethysmograph (Buxco Electronics, USA) and exposed to PBS or increasing acetylcholine (1, 6, 12, 24, 48 and 96 mg/mL) aerosol until Penh values (index of airway resistance) of rats attained 2 times the value (PC-100) seen with PBS alone. The respiratory parameters are recorded online using Biosystem XA software, (Buxco Electronics, USA). Penh, at any chosen dose of acetylcholine is, expressed as percent of PBS response and the using a nonlinear regression analysis PC 100 (2 folds of PBS value) values are computed. Percent inhibition is computed using the following formula:

$$\% Inhibition = \frac{PC {100}_{LPS} - PC {100}_{TEST}}{PC {100}_{LPS} - PC {100}_{PBS}} \times 100$$

### Where,

PC100_{LPS}=PC100 in untreated LPS challenged group
PC100ₜₑₛₜ=PC100 in group treated with a given dose of test compound
PC100_{PBS}=PC100 in group challenged with PBS

Immediately after the airway hyperreactivity response is recorded, animals are sacrificed and bronchoalveolar lavage (BAL) is performed. Collected lavage fluid is centrifuged at 3000 rpm for 5 min, at 4° C. Pellet is collected and resuspended in 1 mL HBSS. Total leukocyte count is performed in the resuspended sample. A portion of suspension is cytocentrifuged and stained with Leishmann's stain for differential leukocyte count. Total leukocyte and Neutrophil counts are expressed as cell count (millions cells mL⁻¹ of BAL). Percent inhibition is computed using the following formula.

$$\% Inhibition = \frac{{NC}_{LPS} - {NC}_{TEST}}{{NC}_{LPS} - {NC}_{CON}} \times 100$$

### Where,

NC_{LPS}=Percentage of neutrophil in untreated LPS challenged group
NC_{TEST}=Percentage of neutrophil in group treated with a given dose of test compound
NC_{CON}=Percentage of neutrophil in group not challenged with LPS
   The percent inhibition data is used to compute ED50 vales using Graph Pad Prism software (Graphpad Software Inc., USA).
   3. In-Vivo Assay to Evaluate Efficacy of MRA in Combination with Corticosteroids Ovalbumin Induced Airway Inflammation:

Guinea pigs are sensitised on days 0, 7 and 14 with 50 μg ovalbumin and 10 mg aluminium hydroxide injected intraperitoneally. On days 19 and 20 guinea pigs are exposed to 0.1% w v⁻¹ ovalbumin or PBS for 10 minutes, and with 1% ovalbumin for 30 minutes on day 21. Guinea pigs are treated with test compound (0.1, 0.3 and 1 mg kg⁻¹) or standard 1 mg kg⁻¹ or vehicle once daily from day 19 and continued for 4 days. Ovalbumin/PBS challenge is performed 2 hours after different drug treatment.

24 hours after the final ovalbumin challenge BAL is performed using Hank's balanced salt solution (HBSS). Collected lavage fluid is centrifuged at 3000 rpm for 5 minutes at 4° C. The pellet is collected and resuspended in 1 mL HBSS. Total leukocyte count is performed in the resuspended sample. A portion of suspension is cytocentrifuged and stained with Leishmann's stain for differential leukocyte count. Total leukocyte and eosinophil counts are expressed as cell count (millions cells mL⁻¹ of BAL). Eosinophil is also expressed as percent of total leukocyte count. % inhibition is computed using the following formula.

$$\% Inhibition = \frac{{Eos}_{OVA} - {Eos}_{TEST}}{{Eos}_{OVA} - {Eos}_{CON}} \times 100$$

Where,
Eos_{OVA}=Percentage of eosinophil in untreated ovalbumin challenged group
Eos_{TEST}=Percentage of eosinophil in group treated with a given dose of test compound
Eos_{CON}=Percentage of eosinophil in group not challenged with ovalbumin.

In-Vivo Assay to Evaluate Efficacy of “MRA” in Combination with p38 MAP Kinase Inhibitors

Lipopolysaccharide (LPS) induced airway hyperreactivity (AHR) and neutrophilia:

### Drug Treatment:

MRA (1 μg/kg to 1 mg/kg) and p38 MAP kinase inhibitor (1 μg/kg to 1 mg/kg) are instilled intratracheally under anesthesia either alone or in combination.

### Method:

Male wistar rats weighing 200±20 gm are used in the study. Rats have free access to food and water. On the day of experiment, animals are exposed to lipopolysaccharide (LPS, 100 μg/mL) for 40 minutes. One group of vehicle treated rats is exposed to phosphate buffered saline (PBS) for 40 minutes. Two hours after LPS/PBS exposure, animals are placed inside a whole body plethysmograph (Buxco Electronics, USA) and exposed to PBS or increasing acetylcholine (1, 6, 12, 24, 48 and 96 mg/mL) aerosol until Penh values (index of airway resistance) of rats attained 2 times the value (PC-100) seen with PBS alone. The respiratory parameters are recorded online using Biosystem XA software, (Buxco Electronics, USA). Penh, at any chosen dose of acetylcholine is, expressed as percent of PBS response and the using a nonlinear regression analysis PC100 (2 folds of PBS value) values are computed. Percent inhibition is computed using the following formula.

$$\% Inhibition = \frac{PC {100}_{LPS} - PC {100}_{TEST}}{PC {100}_{LPS} - PC {100}_{PBS}} \times 100$$

### Where,

PC100_{LPS}=PC100 in untreated LPS challenged group
PC100_{TEST}=PC100 in group treated with a given dose of test compound
PC100_{PBS}=PC100 in group challenged with PBS

Immediately after the airway hyperreactivity response is recorded, animals are sacrificed and bronchoalveolar lavage (BAL) is performed. Collected lavage fluid is centrifuged at 3000 rpm for 5 minutes at 4° C. Pellet is collected and resuspended in 1 mL HBSS. Total leukocyte count is performed in the resuspended sample. A portion of suspension is cytocentrifuged and stained with Leishmann's stain for differential leukocyte count. Total leukocyte and Neutrophil counts are expressed as cell count (millions cells mL⁻¹ of BAL). Percent inhibition is computed using the following formula.

$$\% Inhibition = \frac{{NC}_{LPS} - {NC}_{TEST}}{{NC}_{LPS} - {NC}_{CON}} \times 100$$

### Where,

NC_{LPS}=Percentage of neutrophil in untreated LPS challenged group
NC_{TEST}=Percentage of neutrophil in group treated with a given dose of test compound
NC_{CON}=Percentage of neutrophil in group not challenged with LPS
   The percent inhibition data is used to compute ED50 vales using Graph Pad Prism software (Graphpad Software Inc., USA).
   In-Vivo Assay to Evaluate Efficacy of “MRA” in Combination with β2-Agonists

### Drug Treatment:

MRA (1 μg/kg to 1 mg/kg) and long acting β₂ agonist are instilled intratracheally under anesthesia either alone or in combination.

### Method

Wistar rats (250-350 g) or balb/C mice (20-30 g) are placed in body box of a whole body plethysmograph (Buxco Electronics., USA) to induce bronchoconstriction. Animals are allowed to acclimatize in the body box and are given successive challenges, each of 2 minutes duration, with PBS (vehicle for acetylcholine) or acetylcholine (i.e., 24, 48, 96, 144, 384, and 768 mg/mL). The respiratory parameters are recorded online using Biosystem XA software, (Buxco Electronics, USA) for 3 min. A gap of 2 minutes is allowed for the animals to recover and then challenged with the next higher dose of acetylcholine (ACh). This step is repeated until Penh of rats attained 2 times the value (PC-100) seen with PBS challenge. Following PBS/ACh challenge, Penh values (index of airway resistance) in each rat/mice is obtained in the presence of PBS and different doses of ACh. Penh, at any chosen dose of ACh is, expressed as percent of PBS response. The Penh values thus calculated are fed into Graph Pad Prism software (Graphpad Software Inc., USA) and using a nonlinear regression analysis PC 100 (2 folds of PBS value) values are computed. % inhibition is computed using the following formula.

$$\% Inhibition = \frac{PC {100}_{TEST} - PC {100}_{CON}}{768 - PC {100}_{CON}} \times 100$$

### Where,

PC100_{CON}=PC100 in vehicle treated group
PC100ₜₑₛₜ=PC100 in group treated with a given dose of test compound
768= is the maximum amount of acetylcholine used.

1. A compound having the structure of Formula I: 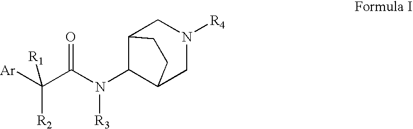
wherein
Ar is aryl or heteroaryl;
R₁ is hydrogen, hydroxy, alkyl, halogen or alkoxy;
R₁ is cycloalkyl, aryl or heteroaryl;
R₃ is hydrogen or alkyl;
R₁ is alkyl, alkenyl, aralkyl or heteroarylalkyl;
or a pharmaceutically acceptable salt, pharmaceutically acceptable solvate, enantiomer, diastereomer, polymorph or N-oxide thereof.

2. A compound selected from:
N-(3-benzyl-3-azabicyclo[3.2.1]oct-8-yl)-N-methyl-2,2-diphenylacetamide (Compound No. 1),
N-3-azabicyclo[3.2.1]oct-8-yl-N-methyl-2,2-diphenylacetamide (Compound No. 2),
N-(3-benzyl-3-azabicyclo[3.2.1]oct-8-yl)-2-hydroxy-N-methyl-2,2-diphenylacetamide (Compound No. 3),
N-(3-benzyl-3-azabicyclo[3.2.1]oct-8-yl)-N-methyl-2,2-diphenylpropanamide (Compound No. 4),
(Exo or Endo) N-(3-benzyl-3-azabicyclo[3.2.1]oct-8-yl)-2-cyclopentyl-2-hydroxy-2-(4-methylphenyl)acetamide (Compound No. 5),
(Exo or Endo) N-(3-benzyl-3-azabicyclo[3.2.1]oct-8-yl)-2-cyclopentyl-2-hydroxy-2-(4-methylphenyl)acetamide (Compound No. 6),
N-(3-benzyl-3-azabicyclo[3.2.1]oct-8-yl)-2-hydroxy-2-(4-methoxyphenyl)-2-cyclopentylacetamide (Compound No. 7),
N-(3-benzyl-3-azabicyclo[3.2.1]oct-8-yl)-2-cyclopentyl-2-hydroxy-2-(4-methoxyphenyl)acetamide (Compound No. 8),
N-3-azabicyclo[3.2.1]oct-8-yl-2-cyclopentyl-2-hydroxy-2-(4-methoxyphenyl)acetamide (Compound No. 9),
N-(3-benzyl-3-azabicyclo[3.2.1]oct-8-yl)-2-chloro-N-methyl-2,2-diphenylacetamide (Compound No. 10),
N-3-azabicyclo[3.2.1]oct-8-yl-2-cyclopentyl-2-hydroxy-2-(4-methylphenyl)acetamide (Compound No. 11),
N-3-azabicyclo[3.2.1]oct-8-yl-2-hydroxy-N-methyl-2,2-diphenylacetamide (Compound No. 12),
(Exo or Endo) N-(3-benzyl-3-azabicyclo[3.2.1]oct-8-yl)-2,2-diphenylacetamide (Compound No. 13),
(Exo or Endo) N-(3-benzyl-3-azabicyclo[32.1]oct-8-yl)-2,2-diphenylacetamide (Compound No. 14),
2-Hydroxy-N-methyl-N-[3-(4-methylpent-3-en-1-yl)-3-azabicyclo[3.2.1]oct-8-yl]-2,2-diphenylacetamide (Compound No. 15),
N-{3-[2-(1,3-benzodioxol-5-yl)ethyl]-3-azabicyclo[3.2.1]oct-8-yl}-2-hydroxy-N-methyl-2,2-diphenylacetamide (Compound No. 16),
N-(3-benzyl-3-azabicyclo[3.2.1]oct-8-yl)-2-hydroxy-2-phenyl-2-(2-thienyl)acetamide (Compound No. 17).

3. A pharmaceutical composition comprising a therapeutically effective amount of a compound of Formula I and one or more pharmaceutically acceptable carriers, excipients or diluents, wherein the compound of Formula I is: 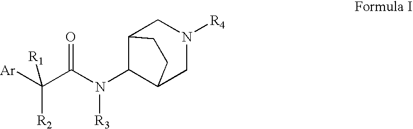
wherein
Ar is aryl or heteroaryl;
R₁ is hydrogen, hydroxy, alkyl, halogen or alkoxy;
R₂ is cycloalkyl, aryl or heteroaryl;
R₃ is hydrogen or alkyl;
R₄ is alkyl, alkenyl, aralkyl or heteroarylalkyl;
or a pharmaceutically acceptable salt, pharmaceutically acceptable solvate, enantiomer, diastereomer, polymorph or N-oxide thereof.

4. A method for the treatment or prophylaxis of a disease or disorder of the respiratory, urinary or gastrointestinal system mediated through the muscarinic receptors comprising administering to an animal or human in need thereof a therapeutically effective amount of a compound of Formula I: 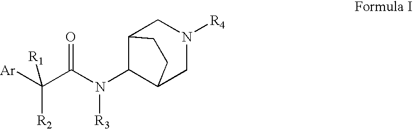
wherein
Ar is aryl or heteroaryl;
R₁ is hydrogen, hydroxy, alkyl, halogen or alkoxy;
R₂ is cycloalkyl, aryl or heteroaryl;
R₃ is hydrogen or alkyl;
R₄ is alkyl, alkenyl, aralkyl or heteroarylalkyl;
or a pharmaceutically acceptable salt, pharmaceutically acceptable solvate, enantiomer, diastereomer, polymorph or N-oxide thereof.

5. The method of claim 4, wherein the disease of disorder is selected from urinary incontinence, lower urinary tract symptoms (LUTS), bronchial asthma, chronic obstructive pulmonary disease (COPD), pulmonary fibrosis, irritable bowel syndrome, obesity, diabetes or gastrointestinal hyperkinesis.

6. (canceled)

7. A pharmaceutical composition comprising a therapeutically effective amount of a compound of Formula I: 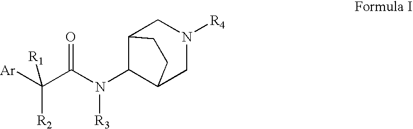
wherein
Ar is aryl or heteroaryl;
R₁ is hydrogen, hydroxy, alkyl, halogen or alkoxy;
R₂ is cycloalkyl, aryl or heteroaryl;
R₃ is hydrogen or alkyl; and
R₄ is alkyl, alkenyl, aralkyl or heteroarylalkyl;
or a pharmaceutically acceptable salt, pharmaceutically acceptable solvate, enantiomer, diastereomer, polymorph or N-oxide thereof;
and one or more therapeutic agent selected from one or more corticosteroids, beta agonists, leukotriene antagonists, 5-lipoxygenase inhibitors, anti-histamines, antitussives, dopamine receptor antagonists, chemokine inhibitors, p38 MAP Kinase inhibitors, PDE-IV inhibitors or a mixture thereof.

8. (canceled)

9. (canceled)
